(19) United States
(12) Patent Application Publication
(10) Pub. No.: US 2024/0024262 A1
(43) Pub. Date: Jan. 25, 2024

(54) USE OF VALERIC ACID IN PREPARATION OF DRUG FOR PREVENTING AND TREATING DIABETES

(71) Applicant: Harbin Medical University, Harbin City (CN)

(72) Inventors: CHANGHAO SUN, HARBIN CITY (CN); TIANSHU HAN, HARBIN CITY (CN)

(21) Appl. No.: 17/898,920

(22) Filed: Aug. 30, 2022

(30) Foreign Application Priority Data
Jul. 21, 2022 (CN) 2022108696129

(51) Int. Cl. A61K 31/19; A61P 3/10

(52) U.S. Cl. CPC A61K 31/19; A61P 3/10

(57) ABSTRACT

The present invention provides a use of valeric acid in preparation of a drug for preventing and treating diabetes and belongs to the technical field of diabetes treatment. The present invention provides a new use of the valeric acid and provides an effect of the valeric acid having a function of preventing and treating type 2 diabetes.

### TECHNICAL FIELD

The present invention belongs to the technical field of diabetes treatment, and particularly relates to a use of valeric acid in preparation of a drug for preventing and treating diabetes.

### BACKGROUND

Partial incidence reasons of diabetes are deficiency of functional pancreatic cells, i.e. insulin-producing islet β cells. The quantity of β cells for type 2 diabetes is reduced by 40-60%, while the quantity of β cells for type 1 diabetes is reduced by 70-97%. Like rodent animals, the quantity of people's pancreatic β cells sharply is increased in a short period of time before and after birth; however, the quantity of matured β cells basically cannot be increased through self-reproduction and proliferation under the natural conditions.

At present, there have been no commercially available drugs capable of inducing proliferation of human islet β cells so far. Ki67 or BrdU experiment results show that the cell proliferation rate of molecules, such as osteoprotegerin and k-aminobutyric acid, for promoting regeneration of human β cells under study is less than 0.5-1%. Therefore, if a drug or product capable of effectively promoting rapid increase in quantity of pancreatic β cells can be researched and developed, it will bring a new hope for prevention and treatment of high-risk diabetes populations, pre-diabetes populations and diabetic patients in the world.

### SUMMARY

In view of this, the objective of the present invention is to provide a use of valeric acid in preparation of a drug for preventing and treating diabetes, and provide a new use of the valeric acid for preventing and treating type 2 diabetes.

In order to achieve the above objective, the present invention provides the following technical solution:

The present invention provides a use of valeric acid in preparation of a drug for preventing and treating diabetes.

Preferably, the diabetes comprises type 2 diabetes.

Preferably, the valeric acid is prepared into valerate, and the valerate is used for treating the diabetes.

Preferably, the valerate comprises sodium valerate.

The present invention further provides a use of valeric acid in preparation of a drug for promoting proliferation of islet β cells.

The present invention has the beneficial effects:

Through in-vitro experiments such as animal experiments and cell experiments, it is found that the valeric acid has a positive action effect on glucose homeostasis by promoting proliferation and differentiation of pancreatic cells, promoting secretion of insulin, improving the sensitivity of insulin and the like. Meanwhile, by comparing the improvement effect of the valeric acid on blood glucose with those of another more extensively researched short chain fatty acid-butyric acid of a similar structure, metformin as a commonly used drug for the diabetes and DYRK1A inhibitor-Harmine having the same effect of promoting proliferation of islet cells, the valeric acid shows a more excellent action effect.

Moreover, in the process of investigating a mechanism of the valeric acid improving blood glucose homeostasis, it is found by proteomics that the valeric acid can up-regulate an expression level of Pdpk1, thereby activating a classical insulin signal pathway PI3K/Akt and then playing roles of inhibiting cell apoptosis, promoting growth proliferation, increasing transportation and uptake of glucose and promoting synthesis of fat and glycogen, which also provides a firm theoretical basis for the action effect of the valeric acid.

### BRIEF DESCRIPTION OF THE DRAWINGS

FIG. 1 is a diagram showing records on food intake;

FIG. 2 is a diagram showing records on body weight;

FIG. 3 is a diagram showing changes on fasting blood glucose;

FIG. 4 is a diagram showing HE staining of mouse pancreatic tissue sections (200 μm);

FIG. 5 is a diagram showing HE staining of mouse pancreatic tissue sections (50 μm);

FIG. 6 is a diagram showing immunohistochemical ERK staining of mouse pancreatic tissues;

FIG. 7 is a diagram showing immunofluorescence insulin (red)/Ki67 (green)/DAPI (blue) of mouse pancreatic tissue sections;

FIG. 8 is a diagram showing immunofluorescence insulin (red)/glucagon/DAPI of mouse pancreatic tissue sections;

FIG. 9 is a diagram showing records on food intake of db/db mice;

FIG. 10 is a diagram showing changes on body weight of db/db mice;

FIG. 11 is a diagram showing changes on blood glucose-related indexes of db/db mice;

FIG. 12 is a diagram showing HE staining of db/db mouse islet tissues;

FIG. 13 is a diagram showing insulin/glucagon immunofluorescence of mouse islet tissues;

FIG. 14 is a diagram showing Ki367/insulin immunofluorescence of db/db mouse islet tissues;

FIG. 15 is a diagram showing experiment results of CCK-8 cell activity;

FIG. 16 is a diagram showing experiment results of EdU-488 cell proliferation, wherein upper: Beta-TC-6 cell line; and lower: mouse primary islet β cells;

FIG. 17 is a diagram showing results of a TUNEL cell apoptosis detection experiment and a flow cytometry experiment, wherein upper: Beta-TC-6 cell line; and lower: mouse primary islet β cells;

FIG. 18 is a diagram showing experiment results of insulin secretion capability, wherein upper: Beta-TC-6 cell line; and lower: mouse primary islet β cells;

FIG. 19 is a diagram showing proteomic results of mouse pancreatic tissues;

FIG. 20 is a diagram showing proteomic results of mouse pancreatic tissue;

FIG. 21 is a diagram showing an insulin signal pathway;

FIG. 22 is a diagram showing expression, verified by WB and ELISA, of differential proteins.

### DESCRIPTION OF THE EMBODIMENTS

The present invention provides a use of valeric acid in preparation of a drug for preventing and treating diabetes. In the present invention, the diabetes preferably comprises type 2 diabetes. In the present invention, the valeric acid is preferably prepared into valerate, and the valerate is used for treating the diabetes. The present invention has no specific restrictions on methods for preparing the valeric acid into the valerate, and those skilled in the art can adopt conventional preparation methods. In the present invention, the valerate preferably comprises sodium valerate. The present invention has no specific restrictions on a dosage form and a preparation method of the drug and a content of the valeric acid in the drug, and those skilled in the art can follow the routine.

In the present invention, the valeric acid has a chemical formula of CH₃(CH₂)₃COOH and a CAS number of 109-52-4, and its structural formula is as follows:

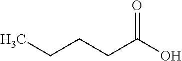

In the present invention, the sodium valerate has a chemical formula of C₅H₉NaO₂ and a CAS number of 6106-41-8, and its structural formula is as follows:

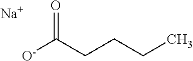

The present invention further provides a use of the valeric acid in preparation of a drug for promoting proliferation of islet β cells. The present invention has no specific restrictions on a dosage form and a preparation method of the drug and a content of the valeric acid in the drug, and those skilled in the art can follow the routine.

Next, the technical solutions provided by the present invention will be described in detail in combination with examples, but cannot be understood as limiting the protective scope of the present invention.

### Example 1

For valeric acid, an intragastric dose for mice is 75 mg/kg/d, which is ⅛ LD50 dose of the valeric acid. The valeric acid was fused with sodium bicarbonate before intragastric administration to prepare a sodium valerate solution until a pH value of the solution was 7.0. According to professional knowledge of pharmacology and toxicology, the daily dose for adults is about 550600 mg/d through conversion.

In this example, a used valeric acid standard with an article number of 240370 and a purity larger than or equal to 99.0% is purchased from Sigma-Aldrich company, and is in a liquid state; and a sodium bicarbonate standard with an article number of S576 and a purity of 99.5-100.5% is purchased from Sigma-Aldrich company, and is in a powder state.

In an animal experiment of high-fat model mice at a first stage, 6-8-week-old male C57BL/6J mice without specific pathogens (SPF grade) are firstly purchased from Liaoning Changsheng Biotechnology Co., Ltd., Experimental animal feeds are ordered from Beijing keaoxieli Feed Co., Ltd., including a mouse AIN-93M maintenance feed and a high-fat feed with a fat energy supply ratio of 45%; the nutrient compositions of the two feeds are in accordance with a rodent experimental animal feed formula recommended by U.S. Department of agriculture; and the AIN-93M maintenance feed is stored at a room temperature, and the high-fat feed with the energy supply ratio of 45% is stored at the low temperature of −20° C.

The animal experimental research was implemented in accordance with the rules and regulations required by Experimental Animal Management Committee of Harbin Medical University, and animals were raised in a laboratory of Clean Experimental Animal Center of School of Public Health of Harbin Medical University. The laboratory conditions are as follows: a temperature is 21.0 (±1.5) degrees Celsius, a relative humidity is 50 (±5)%, lighting is alternately performed for 12:12 hours by using a fluorescent lamp, the feed is freely taken, and distilled water is freely drunk. After being received, 6-8-week-old C57BL6/J mice as a kind of SPF experimental animals were fed in a single cage, and were arranged to freely drink the distilled water and uptake an animal feed for a week of adaptive feeding. Subsequently, the mice were weighed and randomly divided into 4 experimental groups according to the weights of the mice; and the 4 experimental groups were fed with the AIN-93M maintenance feed and the 45% high-fat feed respectively, and were treated with phosphate buffer salt solution (PBS) and different metabolite solutions by oral gavage. A specific grouping manner are as follows: \textcircled{1} ordinary feed feeding group (Control group, fed with the AIN-93M feed); \textcircled{2} 45% high-fat feed PBS gavage group (HFD+PBS group); \textcircled{3} 45% high-fat feed feeding plus valeric acid gavage group (HFD+VA group); \textcircled{4} 45% high-fat feed feeding plus butyric acid gavage group (HFD+BU group), with 12 mice in each group.

During the feeding period of experimental animals, a scattered food amount and a leftover food amount of mice were weighed with an electronic scale and recorded and fresh feeds were changed every day, and a daily food intake was calculated. A appropriate amount of the high-fat feed with the fat energy supply ratio of 45% was transferred from a frozen layer of a refrigerator at −20° C. to a preservation layer at 4° C. every night, and the maintenance feed and the high-fat feed were simultaneously placed at the room temperature for 1 h before each feeding. Fresh distilled drinking water was changed every 3 days. The weights of the mice were weighed and recorded once a week, and a clean animal bedding subjected to high-pressure disinfection was replaced. Mice were intragastrically administrated once a day, and a gavage solvent was freshly prepared before each operation, wherein an experimental dose in a gavage dose is as follows: PBS is given at 5 mg/kg body weight, and the valeric acid and the butyric acid are given at 75 mg/kg body weight respectively; and because the acidity of the valeric acid and the butyric acid was strong, a digestive tract damage is easily caused by direct intragastric administration, the valeric acid and the butyric acid are respectively prepared into the sodium valerate and sodium butyrate for intragastric administration. According to a document “Onyszkiewicz, Maksymilian et al. “Valeric acid lowers arterial blood pressure in rats.” European journal of pharmacology vol. 877 (2020): 173086. doi:10.1016/j.ejphar.2020.173086″, the dose of intravenous injection in each rat is 0.15 mmol/kg. According to conversion of oral and intravenous doses in Pharmacological Experimental Methodology edited by Shuyun Xu and an equivalent dose ratio of rats and mice, a dose of the valeric acid is finally determined as 75 mg/kg which is approximately equal to ⅛ of a half lethal dose of the valeric acid, meeting a safety range in toxicology.

In order to detect a blood glucose homeostasis level and an insulin function of each mouse, an oral glucose tolerance test (OGTT) for the mice was performed. At the night before this test, test mice were fasted but not water for 16 h and transferred from a feeding room to a clean experimental operation room, so that the animals could adapt to the environment. Before the OGTT started, a weight of each mouse was accurately weighed, and an injection dose of glucose was calculated according to 2 mg/g body weight, and a tail was cut for collecting blood at 0 minute, 15 minutes, 30 minutes, 60 minutes and 120 minutes after intragastric injection of the glucose. A first drop of blood after tail cutting was discarded, and then concentrations of blood glucose (mmol/L) were continuously detected using an Accu-Chek blood glucose meter (Roche diagnosis, USA). The test mice were fasted for 12 h at the night before sampling, and were anesthetized by intraperitoneal injection of a 10% chloral hydrate-containing solution. After the intraperitoneal cavities of the mice were cut, blood was collected by apical blood collection, and the test mice were killed. After being collected, blood samples were left for still standing for 2 h at the room temperature, and then centrifuged for 15 min at 300 r/min, serum at the upper layer was sucked, and then biochemical detection was performed immediately. After the blood samples were collected, pancreatic tissues of the mice were immediately cut off so as to prevent pancreatic dissolution, and heads and tails of pancreas were separately stored. Then other tissues of the mice were extracted. Pictures of tissue morphologies of various parts of the mice were photographed and stored, and weights of tissues were weighed and precisely recorded. Subsequently, the extracted tissues were divided into two parts, one of the two parts was sub-packed into an EP tube, immediately rapidly frozen in liquid nitrogen and then transferred to a −80° C. refrigerator for cryopreservation; and the other part was sub-packed into an EP tube containing 4% paraformaldehyde to be fixed for making pathological tissue sections later.

During the subsequent test, in order to observe pathological morphologies of animal tissues, paraffin sections of mouse tissues were made and subjected to HE staining, immunohistochemical staining and immunofluorescence staining. Also, mouse pancreatic tail tissues in a valeric acid intervention group and a control group were taken out from the −80° C. refrigerator and underwent TMT labeled quantitative proteome detection of the animal tissues, and differential proteins were subjected to a Western Blot test and a real-time fluorescence quantitative polymerase chain reaction (QRT PCR) test for the animal tissues to verify test results.

Finally, to detect whether tumor invasion was caused during intervention or not, contents of a carbohydrate antigen (CA 19-9) and a carcinoembryonic antigen (CEA) in mouse serum were detected and compared by using automatic electrochemiluminescence immunoassay system Cobas® 8000 (Roche, USA) and electrochemiluminescence immunoassay (ECLIA).

During the cell testing phase of this example, a cell line of mouse insulinoma islet β cells (Beta-TC-6) and mouse primary islet β cells were purchased from Wuhan Prosser Life Technology Co., Ltd for carrying out relevant experimental researches.

A cell Counting Kit 8 (CCK-8) is a convenient and reliable method to detect the cell activity. In this example, a BeyoClick™ EdU-488 kit is further used to detect the proliferation level of the cells. EdU (5-ethynyl-2′-deoxyuridine) is a thymidine analog that may replace T to infiltrate replicated DNA molecules in the process of cell proliferation. The replication activity of DNA was detected by specific reaction of EdU and an Apollo® Fluor 488 fluorescent dye, an EdU label was correctly detected, and then proliferation of cells was reflected. In this example, expression of Ki-67 in the cells was detected by immunofluorescence (immunocytochemistry), and apoptosis was analyzed by referring to a one-step TUNEL cell apoptosis detection kit (red fluorescence). Meanwhile, this example used a flow cytometry technology to complete detection of a cell cycle and apoptosis.

The following drugs are taken as comparative examples:
1. Comparative example where an existing drug serves as a positive control
(1) High-fat fed C57BL6/J mice
Sodium butyrate gavage intervention group: 75.0 mg/kg
(2) Type 2 diabetes model db/db mice
Sodium butyrate gavage intervention group: 75.0 mg/kg
Harmine gavage intervention group: 10.0 mg/kg
Metformin gavage intervention group: 100.0 mg/kg
(3) Cell line of mouse insulinoma islet β cells (Beta-TC-6)
Sodium butyrate intervention group: 1.0 mmol/L
Harmine intervention group: 10.0 μmmol/L
(4) Mouse islet β primary cell
Sodium butyrate intervention group: 1.0 mmol/L
Harmine intervention group: 10.0 μmmol/L
2. A control test is taken as a comparative example
(1) High-fat fed C57BL6/J mice
Ordinary feed feeding group, and high-fat feed PBS gavage group
(2) Type 2 diabetes model db/db mice
Control feeding group and PBS gavage group
(3) Cell line of mouse insulinoma islet β cells (Beta-TC-6)
Blank control group, and palmitic acid (PA) intervention negative control group
(4) Mouse islet β primary cell
Blank control group, and PA intervention negative control group
Results are as follows:

(I) An Animal Test is Performed to Explore the Influence of the Valeric Acid on Blood Glucose Homeostasis

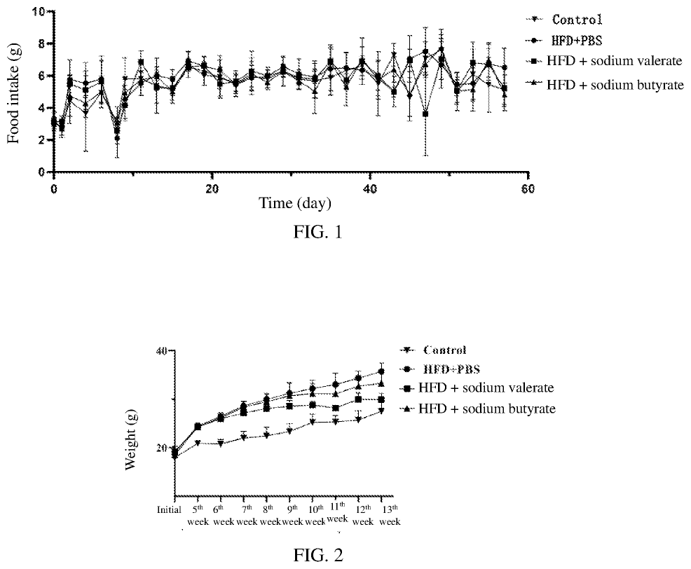

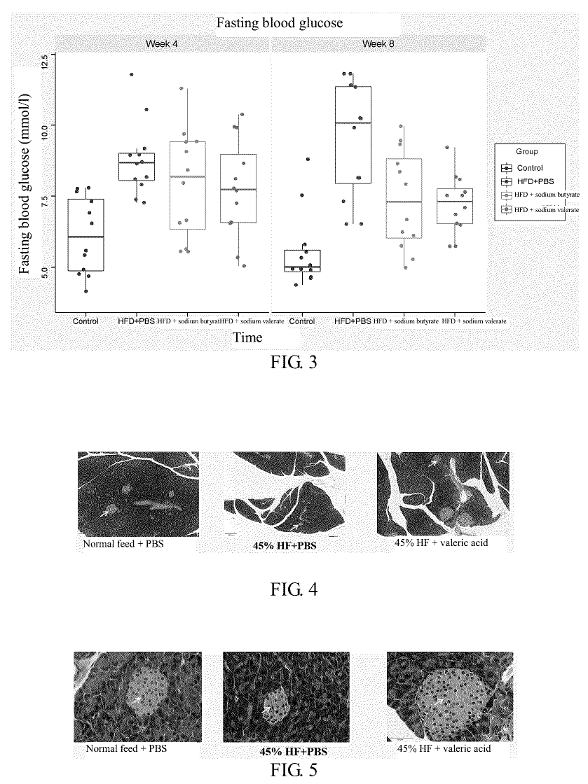

Weights of mice fed with a normal feed (NC group) are increased slightly within 8 weeks, and weights of mice fed with a high-fat feed (HF-PBS group) shows a significant increase trend, but weights of mice under the intervention of the valeric acid (HF-V group) shows a decline trend starting from the fifth week after intervention, and are significantly lower than those in the HF-PBS group after the sixth week (FIG. 1); however, there is no significant difference in food intake among various groups during the whole intervention period (FIG. 2), indicating that weight losses of the mice in HF-V group are not caused by dietary changes of the mice due to the valeric acid. At the time point of 8 weeks of intervention, the fasting blood glucose, fasting insulin contents and areas under OGTT-2h blood glucose curves of the mice are detected, and the above of HF-V group is significantly lower than those of HF-PBS control group, indicating that valeric acid may play an important role in improving blood glucose homeostasis (FIG. 3).

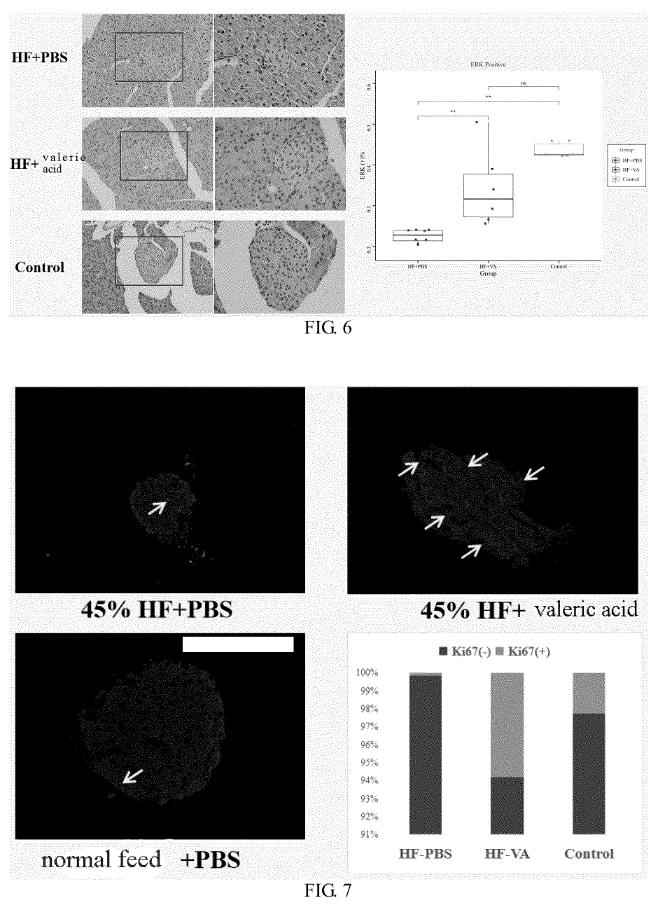

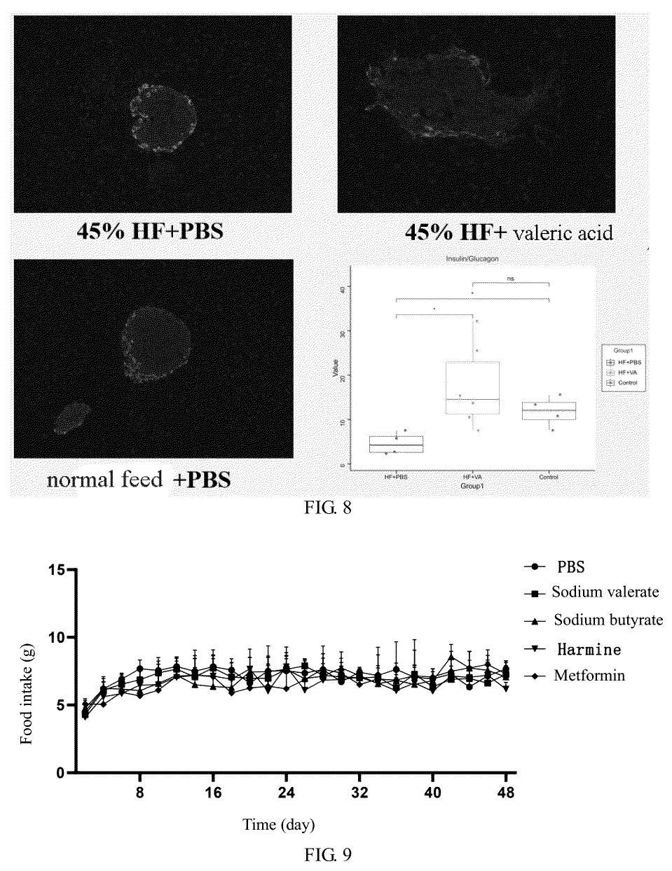

When the histopathological results of mouse pancreas are analyzed, it is found that the islet areas, the number of islets and the number of islet β cells in the HF-PBS group β all become less and smaller compared with those in the control group; but after valeric acid intervention, the islet areas of the mice of the HF-V group are obviously enlarged, the number of islets is increased (FIG. 4), and the number of β cells in the islet is also significantly increased (FIG. 5). In addition, it is found by extracellular regulated protein kinases (ERK) staining on the paraffin sections that a proportion of ERK positive areas is increased significantly, indicating that the pancreatic cells are in proliferative growth and active mitosis (FIG. 6); after insulin/Ki67/DAPI immunofluorescence staining of the pancreatic tissue sections of the mice, it is found that the number of Ki67 stained nuclei in the pancreas of the mice in the valeric acid intervention group is increased significantly, indicating that the number of cells in a proliferative and mitotic phase is increased (FIG. 7); and after insulin/glucagon/DAPI immunofluorescence staining, it is found that a secretion proportion of the insulin in the pancreas of the mice in the valeric acid intervention group is increased significantly, indicating that a function of the islet β cells is up-regulated (FIG. 8).

After it is observed that the valeric acid can promote the mitosis of the islet cells, CEA (carcinoembryonic antigen), CA19-9 (oligosaccharide tumor associated antigen) and other pancreatic cancer specific tumor markers in the mouse serum are detected in order to verify whether tumors are generated or not, so as to find that in HF+PBS and HF+VA mouse serum, CEA <0.200 U/mL, and CA19-9<0.600 U/mL, indicating that cancer invasion does not occur in the mice, and the increase in the number of the islet cells belongs to physiological cell proliferation.

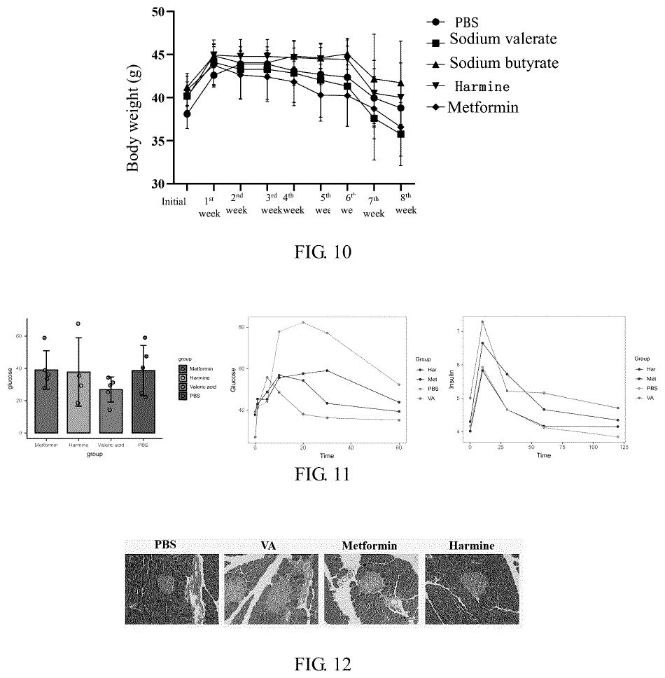

(II) In the animal test, the therapeutic effect of the valeric acid on diabetes is observed by using Leptin receptor gene deficient db/db mice (purchased from Nanjing Junke Biotechnology Co., Ltd.) as test subjects. It is found that there is no significant difference in food intake of test mice in each group (FIG. 9), while weights of the valeric acid group are decreased significantly compared with those of the PBS control group (FIG. 10). It is found especially when blood glucose homeostasis related indicators are detected that compared with the PBS control group, the fasting blood glucose only in the valeric acid group is decreased significantly by nearly 10 mmol/L; a change of the OGTT curve shows that the area under the curve of the valeric acid intervention group is the smallest and significantly better than those of the metformin group and the Harmine positive drug group; and meanwhile, changes on the insulin also show that insulin secretion of the valeric acid is the best, which is superior to the Harmine group, and there was basically no difference between the metformin group and the PBS control group (FIG. 11).

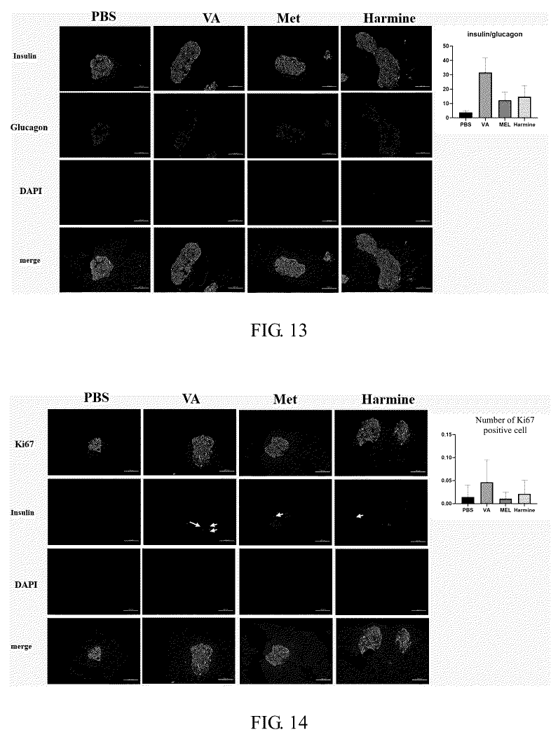

Histopathological examination is also performed on the pancreatic tissues of db/db mice. It is found that the islet area and islet cells of each mouse in the valeric acid intervention group are both increased significantly (FIG. 12), and a ratio of the insulin to glucagon increased significantly, indicating that insulin secretion is significantly improved (FIG. 13). Discovered by staining on the Ki67/insulin, a proportion of Ki67 (+) is increased significantly, indicating that mitosis of the mouse pancreatic cells in the valeric acid intervention group is increased (FIG. 14).

(III) A Cell Test is Performed to Explore the Influence of the Valeric Acid on Blood Glucose Homeostasis

Test results are as follows:

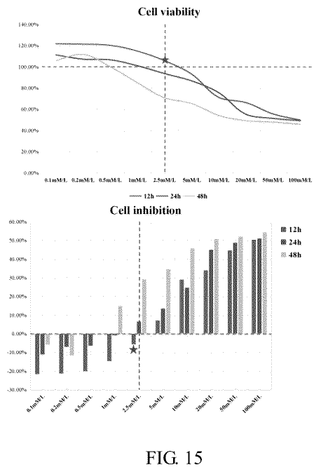

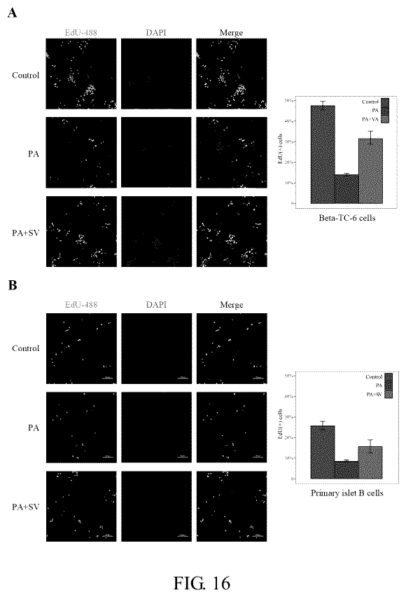

The valeric acid was prepared into sodium valerate, and then the test was performed. In the process of a CCK-8 cell activity test, sodium valerate solutions with gradient concentrations of 0.1 mmol/L, 0.2 mmol/L, 0.5 mmol/L, 1 mmol/L, 2.5 mmol/L, 5 mmol/L, 10 mmol/L, 20 mmol/L, 50 mmol/L and 100 mmol/L (converted to valeric acid concentrations of mmol/L, 0.16 mmol/L, 0.40 mmol/L, 0.8 mmol/L, 2 mmol/L, 4 mmol/L, 8 mmol/L, 16 mmol/L, 40 mmol/L and 80 mmol/L respectively) were used to intervene the Beta-TC-6 cells and the mouse primary islet β cells for 12 h, 24 h and 48 h respectively, the influence effects of the sodium valerate on the activity of the islet cells under different concentrations and different intervention durations were calculated according to detection results of the CCK-8 Kit (FIG. 15). According to the activity level of the cells, it was finally determined that for the sodium valerate, an intervention concentration was 1 mmol/L, and an intervention duration was 12 h.

The Beta-TC-6 cells and the mouse primary islet β cells are detected by the EdU-488 cell proliferation test, it is found that after palmitic acid PA intervention, a proportion of EdU (+) cells is decreased significantly, and the proliferation level of cells is decreased significantly; and after PA intervention, sodium valerate SV intervention is continued on the cells, and the proportion of EdU (+) cells in the cells is increased significantly compared with PA group, and the level of cell proliferation is increased significantly (FIG. 16).

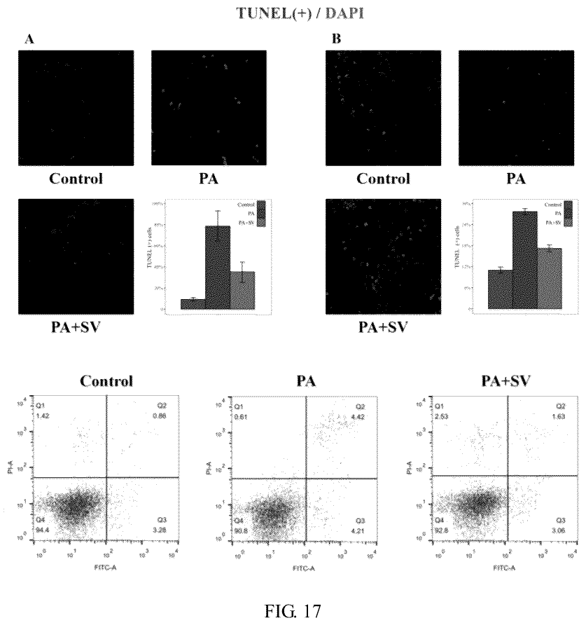

Through a TUNEL cell apoptosis detection test, a proportion of apoptotic cells is increased significantly after PA intervention and slightly decreased after SV intervention. Flow cytometry is used to analyze the Beta-TC-6 cells: after PA intervention, proportions of Q2 quadrant (late apoptosis) and Q3 quadrant (early apoptosis) are increased significantly; after SV intervention, the proportion of apoptotic cells is decreased significantly. Sodium valerate SV can weaken PA induced apoptosis to a certain extent (FIG. 17).

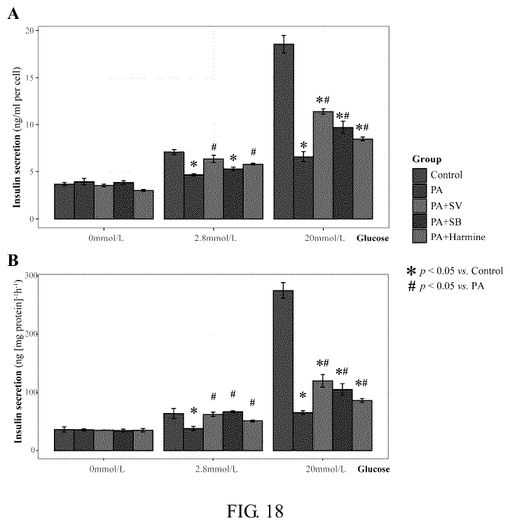

Through an insulin secretion ability test, the levels of the insulin produced in each group under concentrations of no sugar, low sugar (2.8 mmol/L) and high sugar (20 mmol/L) are compared. After PA intervention, the secretion ability of the insulin is decreased significantly; after SV, Sb and Harmine intervention, the secretion levels of the insulin stimulated under a high glucose environment are all significantly increased, and the ability of the sodium valerate to promote secretion of the cell insulin is more significant (FIG. 18).

(IV) Exploration on Mechanism of Valeric Acid to Improve Blood Glucose Level

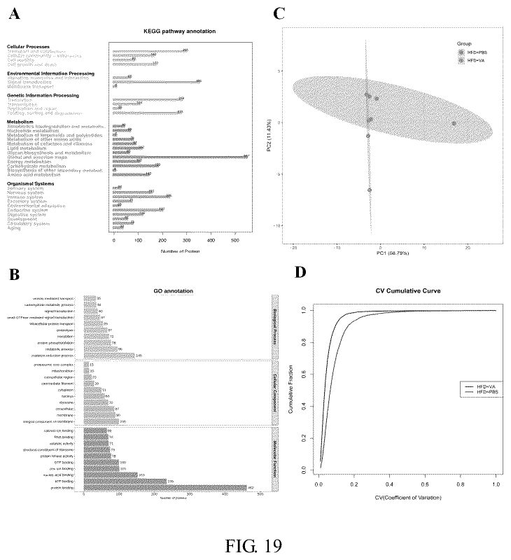

In the process of TMT proteomic detection of the pancreatic tissues in high-fat model mice HFD+PBS and HFD+VA groups, it is found that by PCA analysis, an overall protein difference between the two groups is significant; and CV analysis shows that the two groups have good repeatability. Through functional annotation of GO and KEGG databases, metabolic pathways such as energy and glycolipid metabolism, glycogen synthesis, redox and phosphorylation are enriched (FIG. 19).

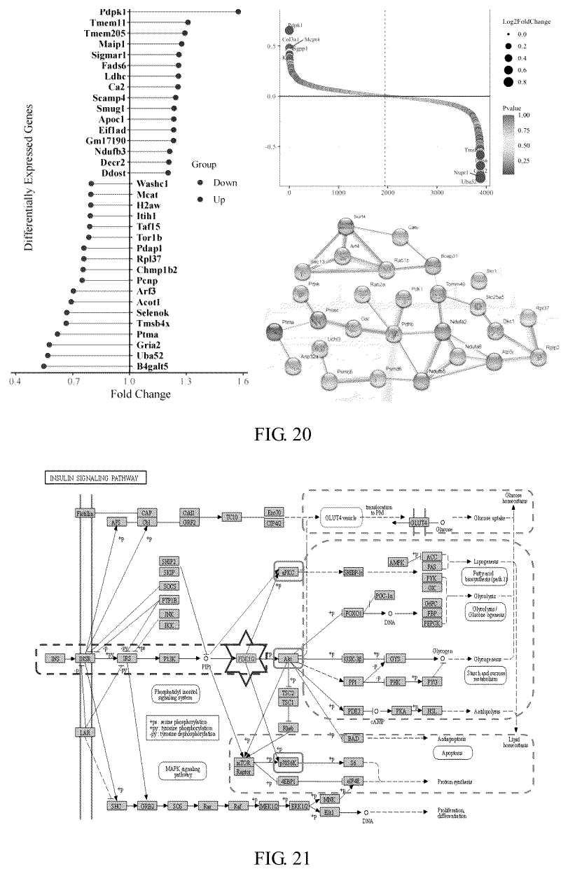

According to the criteria of FC>1.2 or <0.8 and p-FDR<0.05, it is determined that there are differential proteins in the mouse pancreatic tissues in the HFD+PBS group and the HFD+VA group: 16 proteins are up-regulated, and 18 proteins are down-regulated. Through analysis in combination with a interaction network of the differential proteins, differential protein 3-phosphoinositide dependent protein kinase 1 (Pdpk1) with significantly increased expression level in the mouse pancreatic tissues after valeric acid intervention is determined (FIG. 20).

A PI3K (phosphatidylinositol 3-kinase) pathway is the most important insulin metabolism pathway, wherein Pdpk1 plays a core role to activate a protein kinase family, promote utilization and transformation of sugar, inhibit glycogenolysis, mediate a survival pathway of the β cells and promote growth and proliferation of the β cells (FIG. 21).

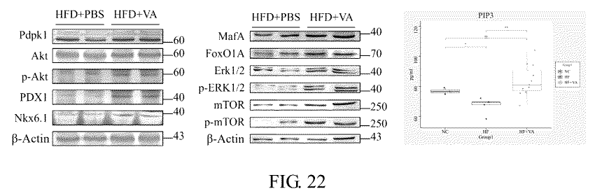

Also, the expression level of key proteins of PI3k/Pdpk1/Akt insulin metabolic pathway in the mouse pancreatic tissues are verified by WB and ELISA: the expression levels of Pdpk1, Akt, p-Akt, mTOR, p-mTOR and PIPS in the mouse pancreatic tissues in the valeric acid group are all significantly increased (p value <0.05); and meanwhile, an expression level of classical proteins in a cell proliferation and differentiation pathway is verified: the expression levels of PDX1, Nkx6.1, MafA, FoxO1A, ERK1/2 and p-ERK1/2 are all significantly increased (FIG. 22).

In conclusion, the valeric acid significantly restores the number of the islet β cells, increases the secretion level of the insulin and reduces blood glucose, thereby exerting the effect of treating the diabetes.

The above descriptions are only preferred examples of the present invention. It should be noted that several improvements and amendments can be made by persons of ordinary skill in the art without departing from the principle of the present invention, and these improvements and amendments are also deemed to the protective scope of the present invention.

1. A use of valeric acid, comprising:
preparing a drug for preventing and treating diabetes by using the valeric acid; and
administering the drug to patients with type 2 diabetes at a daily dose in a range of 550-600 milligrams per day (mg/d) of the valeric acid.

2. (canceled)

3. The use according to claim 1, wherein the valeric acid is prepared into valerate, and the valerate is used for treating the diabetes.

4. The use according to claim 3, wherein the valerate comprises sodium valerate.

5. (canceled)

6. The use of valeric acid according to claim 1, wherein the preparing a drug for preventing and treating diabetes by using the valeric acid comprises:
fusing the valeric acid with sodium bicarbonate before administration to prepare a sodium valerate solution until a pH value of the solution is 7.0 to thereby obtain the drug for preventing and treating diabetes.

7. A use of valeric acid, comprising:
administering a drug comprising the valeric acid to diabetes patients at a target dose.

8. The use of valeric acid according to claim 7, wherein the administering a drug comprising the valeric acid to diabetes patients at a target dose comprises:
administering the drug comprising the valeric acid to the diabetes patients at a daily dose in a range of 550-600 mg/d of the valeric acid.

9. The use of valeric acid according to claim 8, wherein the diabetes patients are patients with type 2 diabetes.
